(19) United States
(12) Patent Application Publication
(10) Pub. No.: US 2025/0366739 A1
(43) Pub. Date: Dec. 4, 2025

(54) SYSTEM FOR ATTACHING AUDIOMETRIC TRANSDUCERS TO A USER'S HEAD AND ITS ADAPTABLE CALIBRATION SYSTEM

(71) Applicant: MY MEDICAL ASSISTANT, REIMS (FR)

(72) Inventor: Nicolas, Paul, Patrice WALLAERT, REIMS (FR)

(73) Assignee: MY MEDICAL ASSISTANT, REIMS (FR)

(21) Appl. No.: 18/870,258

(22) PCT Filed: May 30, 2023
(86) PCT No.: PCT/IB2023/055503

§ 371 Date: Nov. 27, 2024

(30) Foreign Application Priority Data
Jun. 1, 2022 (FR) FR2205290

(51) Int. Cl. A61B 5/12; A61B 5/00

(52) U.S. Cl. CPC A61B 5/121; A61B 5/0051; A61B 5/6803; A61B 5/6815; A61B 2560/0223

(57) ABSTRACT

A system for attaching transducers to the head of a user for performing audiometry using air stimulation and vibratory stimulation includes support systems for two audiometric transducers for air stimulation, each of which can be positioned at the level of one of the auditory pinnae or the external auditory canal, and at least one audiometric transducer for vibratory stimulation, that can be positioned at the level of the desired stimulation zone depending on the chosen conduction mode (for example, one of the mastoids of the skull, the forehead, soft tissues). A method for calibrating vibratory stimulation transducers attached to a support system is described.

The present invention relates to the field of auditory stimulation systems that can be used in audiometry to carry out hearing tests. More specifically, it relates firstly to a method for calibrating a device with audiometric transducers for air stimulation and vibratory stimulation attached to the user's head for performing audiometry, and secondly to a system for holding various devices on the user's head, vibrators and headphones, used in clinical audiometry to bilaterally test a patient's hearing using air stimulation and vibratory stimulation, without moving the headphones during the examination. In other words, it relates to a system for attaching transducers to the user's head for performing air conduction and bone conduction audiometry, as well as a method for calibrating the vibratory stimulation transducer(s) to enable them to be used in conjunction with such an attachment system. Finally, the fixation system makes it possible to eliminate with certainty the contribution of air stimulation emanating from the vibrator and occurring in the high frequencies when only vibratory conduction is to be tested.

A pure-tone audiogram, the hearing test par excellence, comprises two types of audiometry that enable the health of a person's auditory system to be assessed with a sufficient degree of accuracy: air stimulation audiometry, which tests all the structures of the ear (outer, middle and inner ear), and vibratory stimulation audiometry, which tests the inner ear directly using vibrations. These two tests are essential for determining the type of hearing loss (sensorineural loss, conductive loss, mixed loss) of the patient.

In general, an audiogram of this type is performed manually by an operator using an audiometer to which are connected binaural headphones for air stimulation and monaural headphones for vibratory stimulation using exclusively bone conduction (placed on the forehead or the mastoid bone). Contralateral masking of the ear not being tested is also necessary in certain cases, to avoid the possibility of erroneous responses, for example in the event of a significant interaural difference, or in the event of vibratory stimulation for which transcranial transfer is virtually negligible. To carry out the tests, the operator places each of the headphones on the patient's skull either successively when masking is not necessary, or both headphones simultaneously when masking of the contralateral ear is necessary for the vibratory stimulation test. As will be seen in more detail below, the present invention provides a comfortable attachment and fixing system that enables all the transducers required for binaural audiometry using air and/or vibratory stimulation to be positioned correctly and easily on the head without repositioning the transducers and without human intervention.

The choice of the vibrator transducer positioning for vibratory stimulation and/or the type of headphones used for air stimulation impose a specific requirement in terms of transducer calibration, due to the change in ear occlusion induced by the joint presence of the different transducers since, unlike existing solutions, the transducers for air and vibratory stimulation are worn simultaneously on the test ear.

The invention therefore relates to a method of calibrating an audiometric transducer device for air stimulation and vibratory stimulation attached to the user's head for performing audiometry by emitting signals at frequencies below 20 KHz in the direction of the head, consisting of at least one vibratory transducer of the bone conduction vibrator type and an air stimulation apparatus provided with at least one transducer, in a pair of headphones or in the form of an auditory insert, the calibration method consisting of:
Placement of the bone conduction vibrator(s) on the patient's head;
fitting the air conduction headphone/inserts to the patient;
selection of the following parameters:
   one or more tests to be performed among the air stimulation or vibration stimulation tests and, for each of the said tests,
   the ear or ears to be tested,
   the presence or absence of contralateral masking;
selection of the type of transducer used and their positioning;
modification of the calibration of the transducers according to the transducer and the parameters selected, by applying a global frequency weighting to the signals;
the generation by the transducers of frequency signals, constituting the stimuli for vibratory and/or air conduction stimulation.

The principle of frequency weighting is to apply a frequency-dependent change in the intensity of sound to a given value. Noise is measured in dB, with 0 dB representing the audibility threshold. For example, if the measurement gives an unweighted dB value for different frequencies (e.g., 1000, 2000, 4000 Hz . . . ), with a frequency weighting of 10 dB at 1000 Hz, 20 dB at 2000 Hz, and 30 dB at 4000 Hz, the intensity level AFTER weighting will therefore be 10+10=20 dB at 1000 Hz; 10+20=30 dB at 2000 Hz and 10+30 =40 dB at 4000 Hz.

The purpose of weighting is to adapt the intensity level of the sound signal to the condition in which the examination is actually carried out, which differs from the norm (for example, due to the creation of an occlusion effect, or other: see examples below). The overall weighting therefore depends on the type of transducers, their positioning, their coupling, etc.

It is also specified that by overall weighting we mean an accumulation of different weightings which may be applied depending on the context of the test: for a given situation, it is sometimes necessary to sum the weighting coefficients in order to take into account the test situation (see detailed explanations below). The sum of the different weightings is applied to the reference value, provided by the standard, which specifies only one experimental measurement condition (0 dB reference, depending on the type of transducer used for a given positioning, non-occluded test ear).

The side of the head to be tested first for a vibratory stimulation test results from a Weber test including bilateral or frontal bone conduction stimulation at a supraliminal intensity at different audiometric frequencies, for example 0.5 kHz, 1 kHz, 2 kHz, 4 kHz, making it possible to determine the first test side according to the side on which the user perceives the stimulation. This determination is known per se.

In general, the vibration transducer is positioned on the head on at least one location selected from the following:
the front;
the mastoid;
a zone covering a cartilaginous portion of the head.

More specifically:
when stimulation by vibratory transducer takes place on the forehead, the frequency weighting applied is between [−10 and +20] dB;
when stimulation by vibratory transducer takes place on the mastoid, the frequency weighting applied is between [−20 and +10] dB;
when stimulation by vibratory transducer takes place over an area covering a cartilaginous portion of the head, the frequency weighting applied is between [−40; 0] dB.

The additional frequency weighting applied is almost zero, i.e., between [−5; 5] dB if contralateral masking is carried out via the air stimulation transducer placed only on the contralateral ear, and the test ear remains free.

For the record, contralateral masking is obtained by presenting an acoustic message to one ear to prevent it from responding in lieu of the ear being tested.

The frequency weighting applied is between [−30; +10] dB if contralateral masking is performed via the air stimulation transducer placed simultaneously on both ears and the vibration transducer is positioned on the forehead, on the mastoid(s) or on an area covering a cartilaginous portion of the head.

It should be noted that the above weightings can be cumulative, i.e., for a given situation, the weighting coefficients can be added together to take into account the test situation. For example, if the bone conduction test is carried out with the vibrator in the frontal position (weighting A), in the presence of contralateral masking achieved by positioning the air stimulation headphones on both ears simultaneously (weighting B), the overall weighting will be the frequency-by-frequency sum of each of the individual weightings (frequency-by-frequency sum of weighting A, weighting B and weighting C).

The overall weighting coefficient obtained above is intended to cancel out the variations in calibration and reference zero induced by a different positioning of the bone vibrator(s) and air transducer(s) from that prescribed in the standard.

The intensity level required for given experimental conditions for a hearing test using air conduction and bone conduction may be:
Or saved in memory as is, including a calibration table for each of the experimental conditions, the targets of which are determined by applying standard ISO 389-3:2016, to which the global weighting previously calculated is applied (multiple master calibrations). A master calibration is the creation of a calibration table of size n, which contains all the possible experimental combinations (n=occlusion×positioning×masking× . . . ). In short, for each condition, a value is recorded and calibrated. In other words, this amounts to creating as many calibration tables as there could be experimental conditions, with the target values for each table corresponding to the ISO 389-3:2016 value to which the global weighting is applied.
Or calculated prior to stimulus generation, by calling up the intensity level saved in the calibration (ISO 389-3:2016 standard, single parent calibration), to which the global weighting is applied (multiple daughter calibrations). A daughter calibration means that the calibration is performed once (ISO), then the desired output level is calculated from this single calibration by applying the weighting.

In both cases, the result is exactly the same (the global weighting is applied either during the hardware calibration phase or during the stimulus generation phase).

In practice, different application solutions are possible and are given as non-limiting examples. They can be used separately or in combination to guarantee the reliability of the audiometric thresholds obtained during measurements.

In this way, the final stimulation level can be applied:

Either by modifying the intensity level of the transmitted signal, i.e., the target bone stimulation provided by standard ISO 389-3:2016, and applying the global weighting to it (daughter calibration) or by directly calling up the corresponding calibration in the table (mother calibration). For example, if the ISO standard stipulates that a level of 78.9 dB SPL at 500 Hz corresponds to 40 dB HL and the global weighting from the test configuration is −16 dB at this frequency, then the stimulation intensity will be 78.9−16=62.9 dB SPL. The value displayed on the user interface remains 40 dB HL. The target signal presentation level is lowered.

Or by modifying the intensity level of the air masking stimulation produced in the contralateral ear, so as to prevent detection of the bone target signal by the contralateral ear. As a reminder, the intensity of contralateral masking must be between the efficacy criterion (minimum masking intensity necessary to prevent detection of the target sound by the contralateral ear) and the no-overmasking criterion (maximum masking intensity beyond which the masking sound presented in the contralateral ear begins to impact on the detection of the target sound, presented in the test ear). In the present invention, the level of intensity of the masking stimulation can be modified by setting the level of the masking sound at the threshold of the no-overmasking level (provided that the no-overmasking criterion is greater than the efficacy criterion, from which the overall weighting coefficient is subtracted. For example, if the no-overmasking criterion threshold sets the masking noise intensity level at 60 dB HL, and the global weighting is −16 dB at the tested frequency (meaning that the bone conduction detection threshold in the given test conditions improves by 16 dB as compared to the norm), then the masking noise will be 60-(−16) =76 dB HL. Hence, the level of masking noise generated will constrain the detectability of the target sound presented in the test ear and will then modify the threshold obtained by a value equal to the overall weighting.

Or by modifying the value displayed once the measurement result has been obtained by presenting a stimulation intensity level for the target sound and for the masking sound in accordance with the ISO 389-3:2016 standard. The patient's response is simply recorded by taking the value of the target sound and adding the global weighting. For example, if the patient responds at 40 dB HL and the global weighting for the experimental condition tested at the frequency tested is −16 dB, then the patient's response will be recorded at 40-16=24 dB HL.

Any combination of at least two of the three types of application can be used to achieve the same result. For example, if the weighting factor is −16 dB, it is possible to apply −8 dB to the intensity level of the target sound and +8 dB to the intensity level of the masking sound.

As a reminder:

dB HL is the acronym for Decibel Hearing Level, and dB SPL is the acronym for Decibel Sound Pressure Level. In fact, a sound pressure level is expressed in physical decibels (dB SPL). Zero dB SPL is the reference hearing threshold at 1 KHz, obtained from a group of normal subjects. In other words, the acoustic decibel scale would not exist without the human ear. This zero corresponds to a pressure of 20 μPa, the unit of pressure being the Pa (Pascal). Zero Pa can only exist in a vacuum, so this unit cannot be used as an audiometric zero. As the ear is not equally sensitive to all frequencies, it was necessary to express audiometric zeros (the hearing thresholds for each frequency) in dB SPL. For pure-tone audiometry, the measurement of hearing thresholds, i.e., threshold audiometry, is therefore carried out with reference to audiometric zeros. Measurement results are then expressed in dB HL.

As mentioned, the corrective factors or new calibrations are dependent on the acoustic coupling of the transducers, and may vary according to the type of transducers used and their positioning, impacting on the occlusion effect created by the joint presence of the different transducers, made possible by the invention's fixing system.

The introduction of multiple calibrations for bone conduction transducers according to the type of air transducers used must be implemented in order to standardize the thresholds obtained in different experimental conditions that may be encountered (binaural vibratory stimulation headphones worn alone, or alternatively with the addition of binaural air stimulation headphones or ear inserts).

It should be noted that at high frequencies, the effect of occlusion can be negative, i.e., occlusion of the external auditory canal degrades the “vibratory” perception of high frequencies. In fact, occlusion of the auditory canal degrades the perception of the acoustic field generated by the vibrator, which is audible to the subject (air stimulation emitted involuntarily by the vibrator). The support system described below, with the joint presence of the air and vibratory stimulation transducers, prevents the subject from perceiving the air stimulation emitted by the vibrator. This improves the quality of the diagnosis, as the thresholds measured during vibration stimulation reflect the response of the inner ear, including at high frequencies.

At present, the vibratory stimulation transducers used in audiometry all have the same attachment system, i.e., a headband to which a single transducer is attached. No binaural audiometry system using vibration stimulation is currently available, probably because of the various technical problems already mentioned above, which are of course reflected in the transducers placed on the patient's head:

i) Current audiometry systems only offer a single calibration for each transducer, whether for air or vibratory stimulation. The references in ISO 389-3:2016 allow the vibrator to be calibrated for an open ear (not occluded by headphones). The creation of an occlusion induced by the joint presence of the air and vibratory headphones, even partial, significantly modifies the sound level perceived by the patient (and therefore the 0 dB reference used), thus destroying the benefit of the calibration.

ii) The headband is calibrated to provide “sufficient” pressure for audiometry purposes. More precisely, an application force between 4.9 and 5.9 Newtons is normally applied to allow this type of test to be carried out, a force which, however, varies in practice from one individual to another depending on their cranial perimeter. In existing equipment, the clamping force of the headband is never adjusted and is not adjustable. In addition, the existing equipment quickly becomes uncomfortable after only a few minutes' wear, which makes it incompatible with the longer time required to carry out a complete audiometric examination from start to finish.

The pressure at which the transducers are clamped during vibratory stimulation is an important parameter for the physical transmission of signals. Each transducer has a flat, circular surface of 150 to 200 mm² which rests on the patient's head, generally on the mastoid process. When the full clamping force of today's monaural headphones is applied (e.g., when the headband is rigid metal), it causes severe pressure on the mastoid process, and wearing the headphone becomes very uncomfortable and even painful. Fitting the headphones can therefore be tricky and unstable, and the operator must demonstrate a certain amount of skill, especially as it is necessary to reposition the headphones several times in order to test each of the two ears for all tests. As the bone conduction tests have to be carried out with the ear open, the vibrator (ossivibrator on the mastoid and support cushion on the mandible) and the airborne headphones should be positioned diagonally at the same time (i.e., one earphone on the ear to be masked and the other on the cheek, leaving the test ear open and not occluded).

The need to leave one ear open, the rudimentary comfort and the obligation to change the side of the headphones during vibratory stimulation means that it is not possible to carry out all the tests without repositioning the headphones, requiring the presence of an operator to reposition the transducers throughout the various phases of the test. While frontal positioning of the vibrator, mentioned by some authors (e.g., Studebaker 1962), could allow the examination to be carried out without external intervention and modification of the positioning of the headphones, the results obtained in such circumstances for carrying out the examination with vibratory stimulation would be erroneous, in particular because the ossivibrator would have to be calibrated for stimulation with the ear open and not occluded or semi-occluded, unless the headphones was shifted onto the ear not being tested to leave it open.

Until now, these various problems have never been resolved, mainly because there was no need for them, as the tests were carried out face-to-face and the positioning of the headphones could be changed by the experimenter without difficulty. Nowadays, however, the advent of artificial intelligence and telemedicine means that some audiometric tests can be automated and carried out remotely. At least some of the benefits that could be expected from automation and telemedicine (saving human time) are not feasible due to the constraints mentioned above, particularly because of the need for a person to be present when the headphones are put on and off from the patient's skull, or because of the major acoustic impact of a change in the positioning of the vibrators.

The present invention seeks to remedy the above-mentioned disadvantages by offering a comfortable attachment and fixing system that enables all the transducers required for binaural audiometry using air and/or vibratory stimulation to be positioned correctly and easily on the head without repositioning the transducers and without human intervention (without moving the system during the test) during the examination. To meet this objective, more specifically, the invention also presents a more secure system for holding the transducers, as it is better adapted to the individual shape of the human head and ears. The result is improved comfort, enabling the recipient to keep the said invention throughout the examinations without any repositioning needed. The proposed system works in particular with the process of the invention, which is in fact a process for adjusting the initial calibration of the ossivibrator, which makes it possible to compensate for the variations linked to the differences between the actual measurement conditions and the conditions resulting from the 0 dB reference standard (occlusion of the external auditory canal in practice versus open auditory canal in the standard).

In fact, in other words, it must be possible to test both ears binaurally and simultaneously with air and/or vibratory stimulation, with or without contralateral masking, after simultaneous or quasi-simultaneous installation of the necessary transducers (i.e., at least one transducer for vibratory stimulation and two transducers for air stimulation) so that the entire examination can be carried out without external intervention. In the case of a single transducer for vibratory stimulation, the excellent transmission properties of vibratory stimulation through bone (very low transcranial transfer of around 0 to 10 dB, see Appendix 1) or soft tissue are used here to transmit sound bilaterally.

In the case of a single transducer for vibratory stimulation, the vibrator could, for example, be positioned on the skull equidistant from the two ears (for a patient seen from the front: azimuth 0°, i.e., on the frontal bone in the frontal suture axis, or 180° on the occipital bone, in line with the sagittal suture and below the lambdoid suture). In the case of two transducers for vibratory stimulation, the vibrators can, for example, be positioned precisely opposite the mastoid process on either side of the skull. Whatever the number of transducer(s) used for vibratory stimulation, two transducers for air stimulation are added to the previous device, either intra-auricularly (ear inserts) or supra-auricularly (over the ears headphones).

In order to fulfil these objectives, and others which will become clear upon reading this text, the system of the invention, which ensures the attachment of transducers to the user's head for performing audiometry using air stimulation and vibratory stimulation, is such that it comprises means for supporting two air conduction audiometric transducers, each of which can be positioned at the level of one of the auditory pinnae or the external auditory canal, and at least one vibratory stimulation audiometric transducer.

The system in question therefore comprises at least three transducers enabling binaural stimulation for both air stimulation and vibratory stimulation (bone conduction and/or soft tissue). It is therefore designed to stimulate the auditory system for both types of stimulation, enabling auditory functional exploration and providing exhaustive information for a successful diagnosis. It differs from current vibratory stimulation audiometry solutions which allow monaural bone stimulation in compliance with standard NF EN ISO 389-3 or binaural stimulation, but for which the modification of experimental conditions leads to a substantial modification of the equivalent reference levels of threshold vibratory force for pure tone vibrators when the ear is occluded.

Current vibratory stimulation audiometry solutions therefore do not allow binaural bone stimulation without moving the different transducers. As previously mentioned, the support system for the two types of transducers enable the entire test to be carried out without the need to remove them after their initial installation: the said support system must therefore be designed so that, once installed, they guarantee the correct positioning of the transducers and their maintenance over time. Finally, a calibration compensation algorithm makes it possible to avoid the occlusion effect created by the joint presence of different audiometric headsets.

According to one possibility, the support system comprises a combination of a first support system for the audiometric transducers for air stimulation and a second support system for the audiometric transducer(s) for vibratory stimulation. It is up to the user to position them initially, if necessary, one relative to the other, without having to reposition them until the end of the tests.

In fact, according to one possible configuration, the first support system for the audiometric transducers for air stimulation and the second support system for the audiometric transducer or transducers for bone stimulation are fixed to each other, so that there is no need to position them relative to each other. The headset with at least three transducers that they form can in fact be handled in a single piece that needs to be fixed to the head, by adjusting the positions of the transducers in relation to the auditory canal or the auricles on the one hand for air conduction, and the mastoids and the forehead on the other hand for vibratory bone conduction.

In practice, various configurations can be implemented within the framework of the invention. Thus, according to a first example, the second support system for the audiometric transducers in vibratory stimulation may consist of a headband that can be positioned on the forehead (a vibrator) or opposite the mastoid apophyses behind the auricular pinnae. This headband can be flexible or rigid, and its positioning and the force of clamping exerted on the skull can vary at the user's discretion, mainly according to the user's cranial morphology and the location of the auricles. In this case, the vibrator attachment system can be made either by means of a clamping system, using notches provided for this purpose on either side of the vibrator, or simply by using the clamping force of the headband and sliding the ossivibrator under the headband.

Alternatively, the said second support system for the vibratory stimulation audiometric transducers may consist of a semi-rigid cradle surrounding the nape of the neck, the ends of which are fitted with flexible tips placed above the auricular pinnae. Although semi-rigid, the hoop can easily be moved to adapt to the patient's skull.

In general, according to the invention, the first support system for the audiometric transducers for air stimulation consist of a headset comprising an upper headband fitted with means for adjusting its length and either earphones placed on the auricles or inserts inserted directly into the auditory canal. The said headset is positioned in conjunction with the second support system for the vibratory stimulation audiometric transducers described above, for overall positioning of the various transducers.

In certain configurations, the headband may include a branch extending towards the forehead or mastoid bone (bone stimulation) or the pre-tracheal region (soft tissue stimulation) to which an audiometric transducer is attached for vibratory stimulation. This is the one-piece configuration.

Generally speaking, according to the invention, the transducers are fixed to their support system by fixing means that can be adjusted in position in relation to the said support system. It is easy for the user to move them to position them correctly, i.e., in a way that is adapted to his skull and, on each side, to the location of the auricle or external auditory canal and the areas of vibratory stimulation (non-limited to the mastoid bone, forehead, soft tissues of the skull, etc.).

The choice of transducer vibrator positioning for vibratory stimulation and/or the type of headphones used for air stimulation impose particular requirements in terms of transducer calibration, due to a change in ear occlusion induced by the joint presence of the different transducers-since, unlike existing solutions, air and vibratory stimulation are worn simultaneously.

Other aims and advantages of the present invention will become apparent during the description which follows, referring to embodiments which are given only as indicative and non-exhaustive examples.

This description will be easier to understand if reference is made to the attached drawings in which:

FIG. 1 shows a schematic profile view of a user equipped with support system consisting of a headband for vibratory stimulation audiometric transducers (bone and soft tissue conduction).

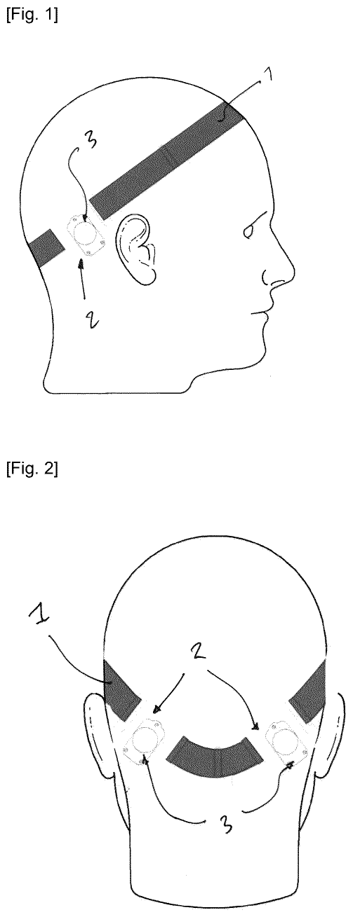

FIG. 2 shows a schematic rear view of the user wearing the support headband.

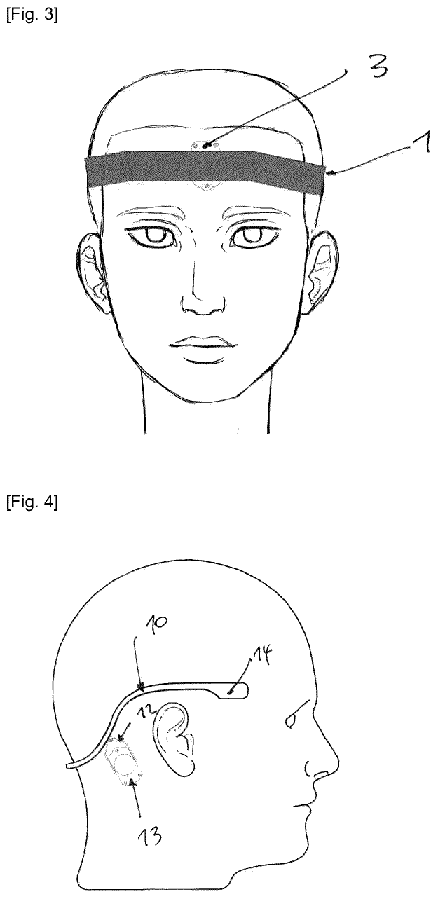

FIG. 3 shows a schematic front view of the user equipped with the said support headband when a single vibrator is used, but positioned frontally.

FIG. 4 shows a schematic profile view of a user equipped with support system, also for vibratory stimulation audiometric transducers, consisting of a semi-rigid hoop surrounding the nape of the user's neck.

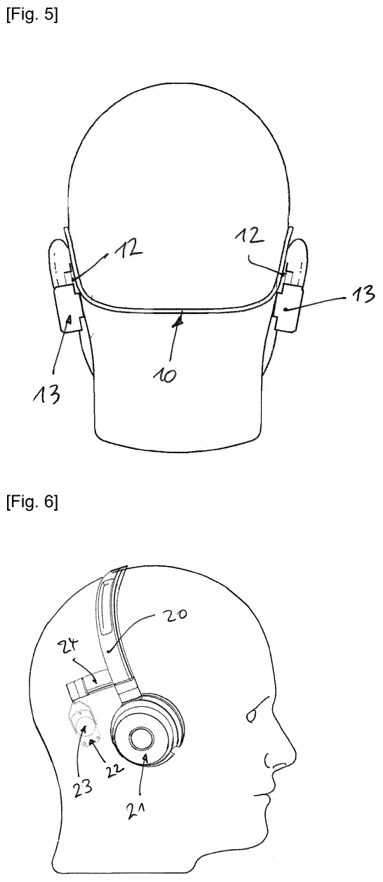

FIG. 5 shows a schematic rear view of the user equipped with the said support bar.

FIG. 6 shows a schematic profile view of a user wearing a combined headset, with an upper headband for the air-stimulation audiometric transducers (earphones) and support arms for the vibration-stimulation audiometric transducers.

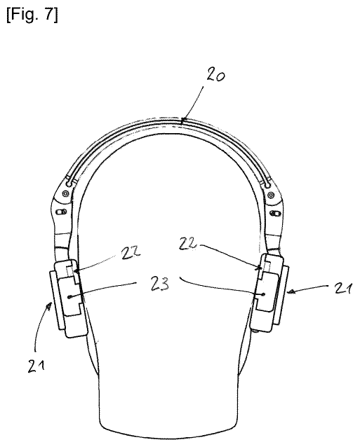

FIG. 7 shows a schematic rear view of the user wearing the combined headset shown in the previous figure.

The configuration in FIG. 1 to FIG. 30 shows a flexible or rigid headband. This particular support is designed to leave the patient's auricles clear so that headphones or binaural air stimulation inserts can be added to complete the audiometric tests. Its nature and positioning mean that it can be installed simultaneously with conventional headphones fitted with earphones or inserts. According to one example, the headband 1 can include fixing system 2 for the vibratory simulation transducer(s) 3, which ensure that the transducer(s) are held stationary with the headband 1. The vibratory stimulation transducer(s) 3 can also be slid under the headband, without any fixing system (see FIG. 3), in which case they are held in place by the pressure force of the headband. These fixing support system 2 can be adjusted/mounted onto the headband so as to modify, for fitting purposes, the distance between the transducer(s) 3 so that they are correctly positioned, generally opposite the stimulation zone (for example the mastoid process of the temporal bone, the forehead or possibly soft tissue), and/or-in the case of a flexible headband-the clamping force exerted by the system.

In this respect, one or more systems for adjusting the clamping force exerted can be implemented: these systems can, for example, consist of slides, press studs, adjustment notches, etc. These adjustment systems make it possible in practice to adjust the pressure exerted by the vibratory stimulation transducers on the patient's bone or soft tissue. In practice, these adjustment systems make it possible to adjust the pressure exerted by the vibratory stimulation transducers on the patient's bone or soft tissue.

The size of the headband can be universal or adjustable and, in the case of a flexible headband 1, the pressure force can also be adjusted by measuring the patient's head circumference using a measuring tool. The result of the measurement is used to fine-tune the diameter of the headband using the adjustment systems and marks positioned on the headband until the required pressure force is reached, corresponding to the measured head circumference. The headband 1 may also include an opening system (slide, clips, hook-and-loop fastener, etc.) to make it easier to place on the patient.

The support systems illustrated in FIG. 4 and FIG. 5 comprise a semi-rigid hoop 10 designed to be positioned behind the patient's head. As with the headband configuration 1 of FIG. 1 to FIG. 3, these support systems are designed to leave the recipient's auricles clear so that headphones or inserts can be attached, the latter constituting the transducers for binaural air stimulation. In practice, the positioning of the rear cradle 10 means that these second support systems can be installed simultaneously with a traditional air stimulation arrangement.

In this case, the fastening system consists of a semi-rigid hoop 10, which can be malleable so that it can be adjusted to the patient's morphology, to which various elements are added. This C-arm 10 may be proposed in different sizes. The headband 10 should be placed just above the earlobes and should follow the contour of the head. One or more fixing systems 12 are used to fix the vibrator or vibrators 13 constituting the vibratory stimulation transducers to the hoop 10. These fixing systems 12 can be moved along the axis of the cradle 10 to enable them to be positioned opposite the mastoid process of the temporal bone or possibly the pretragal area for soft tissue stimulation. Two flexible endpieces 14 hold the assembly in position. The flexible tips 14 must rest slightly in front of the ear so as to be comfortable while maintaining support for the vibrators 13 on the stimulation zone.

A third combined configuration is shown in FIG. 6 and FIG. 7, in which the support systems are based on a headset fitted with air stimulation headphones 21. The device of the invention consists of an upper cradle 20, which in fact constitutes the support systems and can be telescopic, i.e., comprise extendable arms for adaptation to the size of each patient's head. The extendable arms of the upper cradle 20 are terminated at each end by a professional earphone 21 for air conduction and by an audiometric vibrator 23 for bone conduction. Another example of the invention would be to have one or more other extendable arms of the upper cradle oriented in the direction of the chosen stimulation zones (e.g., forehead, mastoids, pre-tracheal zones, etc.). The vibrators 23 making up the vibratory simulation transducers are placed on fixing system 22 arranged at the free end of branches 24 connecting them to the arms of the upper arch 20.

In all the cases mentioned, the transducers are intended to be connected to a conventional audiometer by wired connectors or by wireless signal transmission technology.

The above examples are not limiting of the invention, which on the contrary encompasses the variants of shape and configuration that are within the scope of the invention.

1. Method A method for calibrating an audiometric transducer device for air stimulation and vibratory stimulation attached to the user's head for performing audiometry by emitting signals at frequencies below 20 kHz in the direction of the head, comprising at least one vibratory transducer of the bone conduction vibrator type and an air stimulation apparatus provided with at least one transducer, in a pair of headphones or in the form of an auditory insert, the calibration method consisting of:
placement of the bone conduction vibrator(s) on the patient's head;
fitting the air conduction headphone/inserts to the patient;
selection of the following parameters:
one or more tests to be carried out using air stimulation or vibratory stimulation and, for each of these tests;
the ear(s) to be tested; and
the presence or absence of contralateral masking;
selection of the type of transducer used and their positioning;
modification of the transducer calibration according to the transducer and the parameters selected, by applying a global frequency weighting to the signals; and
generation by the transducers of frequency signals, constituting the stimuli for vibratory and/or aerial stimulation.

2. The method of calibrating an audiometric transducer device according to claim 1, wherein the vibratory transducer is placed on at least one location of the head selected from the following:
the forehead or on the median axis of the skull;
the mastoid;
a zone covering a cartilaginous portion of the head.

3. The method of calibrating an audiometric transducer device according to claim 1, wherein:
when stimulation by vibratory transducer takes place on the forehead, the frequency weighting applied is between [−10 and +20] dB;
when stimulation by vibratory transducer takes place on a mastoid, the frequency weighting applied is between [−20 and +10] dB;
when stimulation by vibratory transducer takes place over an area covering a cartilaginous portion of the head, the frequency weighting applied is between [−40 and 0] dB.

4. The method for calibrating an audiometric transducer device according to claim 1, wherein the additional frequency weighting applied is virtually zero, i.e., between [−5; 5] dB if contralateral masking is performed via the air stimulation device placed on the contralateral ear only, and the test ear is unoccluded.

5. The method for calibrating an audiometric transducer device according to claim 1, wherein the frequency weighting applied is between [−30; +10] dB if contralateral masking is performed via the air stimulation device placed simultaneously on both ears and the vibratory transducer is positioned on the forehead, on the mastoid(s) or on a zone covering a cartilaginous portion of the head.

6. A system for attaching transducers to the head of a user for performing audiometry using air stimulation by air conduction and vibratory stimulation by bone conduction or by conduction in soft cartilaginous tissues, wherein it comprises support systems for two audiometric transducers for air stimulation, each of which can be positioned at one of the auditory pinnae or the external acoustic meatus, and at least one audiometric transducer for bone stimulation, which can be positioned onto at least one desired stimulation zone selected from one of the mastoids of the skull, the forehead, and a zone covering a cartilaginous portion of the head.

7. The fixing system according to claim 6, wherein the support system comprises a combination of first support system for the audiometric transducers for air stimulation and second support system for the audiometric transducers for vibration stimulation.

8. The fixing system according to claim 7, wherein the first support system of the audiometric transducers for air stimulation and the second support system of the audiometric transducers for vibration stimulation are connected to each other.

9. The fixation system according to claim 8, wherein the second support system for the audiometric transducer or transducers in vibratory stimulation consist of a headband which can be positioned above or below the auricles.

10. The fixation system according to claim 8, wherein the second support system for the audiometric transducer or transducers in vibratory stimulation consist of a semi-rigid hoop surrounding the nape of the neck and the ends of which are provided with flexible tips placed above the auricular pinnae.

11. The fixation system according to claim 8, wherein the first support system for the audiometric transducers for air stimulation consist of a headset comprising an upper headband equipped with means for adjusting its length and earphones placed on the auricular pinnae or in the external auditory canal.

12. A fastening system according to claim 11, wherein the headband comprises, either in the vicinity of each earphone, or at its base, one or more branches extending in the direction of the vibratory stimulation zones (for example, mastoid bone, forehead, soft tissues) to which an audiometric transducer is attached for vibratory stimulation.

13. The fixing system according to claim 6, wherein the transducers are fixed to their support systems via fixing means whose position is adjustable relative to said support systems.

14. The method of calibrating an audiometric transducer device according to claim 2, wherein:
when stimulation by vibratory transducer takes place on the forehead, the frequency weighting applied is between [−10 and +20] dB;
when stimulation by vibratory transducer takes place on a mastoid, the frequency weighting applied is between [−20 and +10] dB;
when stimulation by vibratory transducer takes place over an area covering a cartilaginous portion of the head, the frequency weighting applied is between [−40 and 0] dB.

15. The method for calibrating an audiometric transducer device according to claim 2, wherein the additional frequency weighting applied is virtually zero, i.e., between [−5; 5] dB if contralateral masking is performed via the air stimulation device placed on the contralateral ear only, and the test ear is unoccluded.

16. The method for calibrating an audiometric transducer device according to claim 3, wherein the additional frequency weighting applied is virtually zero, i.e., between [−5; 5] dB if contralateral masking is performed via the air stimulation device placed on the contralateral ear only, and the test ear is unoccluded.

17. The method for calibrating an audiometric transducer device according to claim 2, wherein the frequency weighting applied is between [−30; +10] dB if contralateral masking is performed via the air stimulation device placed simultaneously on both ears and the vibratory transducer is positioned on the forehead, on the mastoid(s) or on a zone covering a cartilaginous portion of the head.

18. The method for calibrating an audiometric transducer device according to claim 3, wherein the frequency weighting applied is between [−30; +10] dB if contralateral masking is performed via the air stimulation device placed simultaneously on both ears and the vibratory transducer is positioned on the forehead, on the mastoid(s) or on a zone covering a cartilaginous portion of the head.

19. The method for calibrating an audiometric transducer device according to claim 4, wherein the frequency weighting applied is between [−30; +10] dB if contralateral masking is performed via the air stimulation device placed simultaneously on both ears and the vibratory transducer is positioned on the forehead, on the mastoid(s) or on a zone covering a cartilaginous portion of the head.

20. The fixation system according to claim 9, wherein the second support system for the audiometric transducer or transducers in vibratory stimulation consist of a semi-rigid hoop surrounding the nape of the neck and the ends of which are provided with flexible tips placed above the auricular pinnae.
